(19) United States
(12) Patent Application Publication
(10) Pub. No.: US 2025/0275716 A1
(43) Pub. Date: Sep. 4, 2025

(54) SENSORIZED WEARABLE GARMENT

(71) Applicants: Massachusetts Institute of Technology, Cambridge, MA (US); The General Hospital Corporation, Boston, MA (US)

(72) Inventors: Joseph J. DelPreto, Croton-on-Hudson, NY (US); Daniela L. Rus, Weston, MA (US); Alphonse Taghian, Westwood, MA (US); Cheryl Brunelle, Winchester, MA (US)

(73) Assignees: Massachusetts Institute of Technology, Cambridge, MA (US); The General Hospital Corporation, Boston, MA (US)

(21) Appl. No.: 18/857,598

(22) PCT Filed: Mar. 31, 2023
(86) PCT No.: PCT/US2023/065207

§ 371 Date: Oct. 17, 2024

Related U.S. Application Data
(60) Provisional application No. 63/333,719, filed on Apr. 22, 2022.

(51) Int. Cl. A61B 5/00; A41D 1/00; A41D 31/04; A61F 13/08

(52) U.S. Cl. CPC A61B 5/4833; A41D 1/005; A41D 31/04; A61B 5/0022; A61B 5/6804; A61B 5/7267; A61F 13/08; A61B 2562/0247; A61B 2562/168

(57) ABSTRACT

Described herein are systems and methods for improving the performance of and compliance with wearable garments. In some embodiments, a system comprises one or more sensors integrated within a wearable garment. The sensors are configured to generate data responsive to pressure exerted by the wearable garment on a user; and one or more processors coupled to the one or more sensors. The processors are configured to process the generated data to determine a duration of use of the wearable garment by the user; and in response to determining that the duration of use is less than a predetermined duration of use threshold, generating one or more notifications.

### BACKGROUND

Breast cancer-related lymphedema (BCRL) is a potentially irreversible swelling of the limbs that affects countless women. BCRL is a chronic and progressive disease characterized by swelling in the arm, hand, or trunk on the side of breast cancer treatment, which may progress to an irreversible stage. As of 2021, there are more than 3.8 million women in the United States alone who have been treated for breast cancer (BC); approximately 20% of whom live with BCRL. BCRL treatment aims to prevent a potentially irreversible swelling of limbs due to causes such as breast cancer surgeries. Main risk factors for BCRL, sometimes referred to as lymphedema, include lymph node surgery, elevated body mass index, and regional lymph node radiation.

Patients with BCRL experience a variety of symptoms including heaviness, fullness, and discomfort in the affected limb and may suffer from lower quality of life than those without BCRL. BCRL is a progressive, burdensome condition characterized by lymphatic damage after axillary surgery or lymph node radiation, leading to fluid buildup and swelling on the side of BC treatment. Compression sleeves are the state of the art for treatment of BCRL, but many open questions remain regarding effective pressure and usage prescriptions.

Compression therapy is the mainstay of modern lymphedema management, as preliminary studies suggest short-term compression can be beneficial. Compression sleeves reduce swelling by providing a gradient pressure that decreases from the wrist to the upper arm. Physiologically, the sleeve reduces BCRL by increasing interstitial pressure and tissue fluid drainage, stimulating lymphatic contractions and breaking down fibrosclerotic tissue caused by chronic BCRL. The compression sleeves are prescribed as either USA Class I or Class II. Class I with target pressure gradients of 20≡30 mmHg and Class II with target pressure gradients of 30≡40 mmHg. These pressures have been shown to counteract hydrostatic venous pressure in the arm in the upright position without impeding the lymphatic pump.

### SUMMARY

As wearable devices become more accessible and computationally powerful, they unlock new potential for personalized health care. Continuously monitoring a user's activity and the wearable devices' performance can deliver real-time analytics to patients and caregivers that enable more responsive treatments and a more engaging patient experience. On-device and cloud-based computing can leverage machine learning to extract insights from continuous data streams. One such path to utilize on-device and cloud based computing is to leverage unobtrusive continuous monitoring to improve the understanding of human behavior and the wearable device.

Medical applications of wearable devices and their personalized data streams can lead to more accurate diagnoses and more effective, customizable treatments. Smart wearable devices have great potential to change how technology is integrated into daily life. A particularly impactful and growing application is continuous medical monitoring; being able to stream physiological and behavioral information creates personalized datasets that can lead to more tailored treatments, diagnoses, and research.

Tactile information in particular can provide valuable insights about how humans interact with their environment or about the performance of assistive devices. However, integrating sensors into a wearable garment poses significant challenges. The sensors must accurately detect pressure on soft deformable human skin, even over long periods including various activities, postures, and ambient conditions. For safety and comfort, sensors must be small and conformal so that a sensorized garment is no more obtrusive than the original. Widespread adoption also necessitates minimizing calibration or maintenance by the user, and scalable fabrication. There are significant challenges to developing sensors for high-pressure applications on the human body, including operating between soft compliant interfaces, being safe and unobtrusive, and reducing calibration for new users. One such wearable device is a compression garment. Compression garments provide a gradient pressure which is tightest at one point on the body and decreases proximally, thereby reducing swelling and preventing further lymph accumulation. The gradient pressure reduces swelling in the user and may prevent further lymph accumulation.

Compression garments are available in multiple sizes and lengths, which aim to impart the prescribed pressure on varying limb sizes and shapes. Users are fitted by a trained healthcare professional and instructed to wear the garment for at least 12 hours per day. However, many users may not adhere to this prescription since sleeves may be perceived as aesthetically displeasing or uncomfortable. Often studies analyzing the effect of compression do not typically report adherence data regarding garment-wearing or may rely on patients' self-reports. In some studies, reported adherence has been poor; for example, one study found that only 39≡41% of patients reported wearing their compression garments at least 75% of the prescribed time. In addition, the pressure achieved by a sleeve may not match the expected range.

Described herein is a wearable garment with one or more integrated sensors configured to collect data. The sensors are coupled with a controller and are configured to continuously process the data and to store said data in a cloud-based storage system. The wearable garment may feature one or more pressure sensors, such as pneumatic or resistive sensors, unobtrusively integrated into the fabric of the wearable garment. Alternatively, other sensors may be used. The sensors may be used to measure when the wearable garment is on the user and the pressure exerted by the fabric on the user, other types of data on the user and the wearable garment may be collected. Further, assorted electronics may be incorporated to perform data management and communication.

By leveraging advances in soft sensing to design and deploy an integrated wearable system for continuous pressure monitoring, said system can provide vital information about BCRL treatment. The goal of the wearable garment is to enable long-term, unobtrusive, nearly plug-and-play sensing of whether the wearable garment is being worn and what pressure gradient is being exerting on the arm. This can lead to deployments that improve patient care by yielding insights and verifications about effective wearable garment prescriptions.

According to one aspect of the present disclosure, a system comprises: one or more sensors integrated within a wearable garment, the sensors configured to generate data responsive to pressure exerted by the wearable garment on a user; and one or more processors coupled to the one or more sensors and configured to: process the generated data to determine a duration of use of the wearable garment by the user; and in response to determining that the duration of use is less than a predetermined duration of use threshold, generating one or more notifications.

In some embodiments, the duration of use threshold can be prescribed by a physician. In some embodiments, the wearable garment can be a garment prescribed to the user for treatment of cancer-related lymphedema (BCRL). In some embodiments, the wearable garment can include compression fabric. In some embodiments, the compression fabric can provide a pressure gradient of between 10 mm Hg to 40 mm Hg. In some embodiments, the compression fabric can provide a pressure gradient of between 20 mm Hg to 30 mm Hg. In some embodiments, the compression fabric can compression fabric provides a pressure gradient of between 30 mm Hg to 40 mm Hg. In some embodiments, the wearable garment comprises a sleeve, vest, headband, glove, or sock.

In some embodiments, the one or more sensors can comprise one or more pressure sensors. In some embodiments, the one or more sensors can comprise one or more resistive sensors. In some embodiments, the one or more sensors can comprise one or more pneumatic sensors. In some embodiments, at least one of the one or more pneumatic sensors can comprise an inflatable bladder and a pressure transducer. In some embodiments, the one or more sensors can include multiple sensors arranged along a length of the wearable garment. In some embodiments, at least one of the one or more processors can be integrated with the wearable garment. In some embodiments, at least one of the one or more processors can be physically separate from the wearable garment. In some embodiments, at least one of the one or more processors can be configured to transmit the generated data to an application of a cloud computing environment. In some embodiments, processing the generated data to determine the duration of use of the wearable garment can be based on comparing the generated data to one or more sensor thresholds associated with the one or more sensors. In some embodiments, at least one of the one or more processors can be configured to determine the sensor thresholds based on an analysis of other data generated by the one or more sensors. In some embodiments, at least one of the one or more processors are configured to process the generated data using machine learning (ML). In some embodiments, the one or more processors can be configured to process the generated data to evaluate performance of the wearable garment.

According to another aspect of the present disclosure, a system for detecting use and performance of wearable garment comprises: one or more sensors integrated within with wearable garment; a controller coupled to at least one of the one or more sensors via respective ones of one or more conductive paths and configured to: collect data generated by the one or more sensors; and transmitting the collected data to a remote computing device, wherein the remote computing device is configured to generate one or more metrics related to usage and performance of the garment based on the collected data and to display the metrics.

In some embodiments, the one or more sensors can comprise one or more pressure sensors. In some embodiments, the one or more sensors can comprise one or more resistive sensors. In some embodiments, the one or more sensors can comprise one or more pneumatic sensors. In some embodiments, the one or more sensors can include multiple sensors disposed along a length of the wearable garment. In some embodiments, the wearable garment can comprise a compression fabric. In some embodiments, the wearable garment can comprise a sleeve, vest, headband, glove, or sock.

According to another aspect of the present disclosure, a system comprises one or more sensors integrated within a wearable garment, the sensors configured to generate data responsive to pressure exerted by the wearable garment on a user; and one or more processors coupled to the one or more sensors and configured to process the generated data to evaluate performance of the wearable garment.

### DESCRIPTION OF THE SEVERAL VIEWS OF THE DRAWINGS

The manner and process of making and using the disclosed embodiments may be appreciated by reference to the figures of the accompanying drawings. It should be appreciated that the components and structures illustrated in the figures are not necessarily to scale, emphasis instead being placed upon illustrating the principals of the concepts described herein. Like reference numerals designate corresponding parts throughout the different views. Furthermore, embodiments are illustrated by way of example and not limitation in the figures, in which:

FIG. 1 is a process diagram showing how data collected from a wearable garment can be used to monitor and improve performance and compliance, according to embodiments of the present disclosure;

FIG. 2a shows the outside of a wearable garment with resistive sensors, according to an embodiment of the present disclosure;

FIG. 2b shows the inside of the wearable garment of FIG. 2a;

FIG. 3a is a top view of a resistive sensor, according to an embodiment of the present disclosure;

FIG. 3b is another top view of the resistive sensor of FIG. 3a, here shown partially disassembled;

FIG. 4a shows the outside of a wearable garment with pneumatic sensors, according to an embodiment of the present disclosure;

FIG. 4b shows the inside of the wearable garment of FIG. 4a;

FIG. 5 shows a portion of a pneumatic sensor, according to an embodiment of the present disclosure;

FIG. 6 is a diagram of a system for monitoring and enforcing compliance with a wearable garment, according to an embodiment of the present disclosure;

FIG. 7 is a graph illustrating how data obtained from a sensorized wearable garment can be used to detect performance thereof and compliance therewith, according to some embodiments; and

FIG. 8 is another graph illustrating how data obtained from a sensorized wearable garment can be used to detect performance thereof and compliance therewith, according to some embodiments.

### DETAILED DESCRIPTION

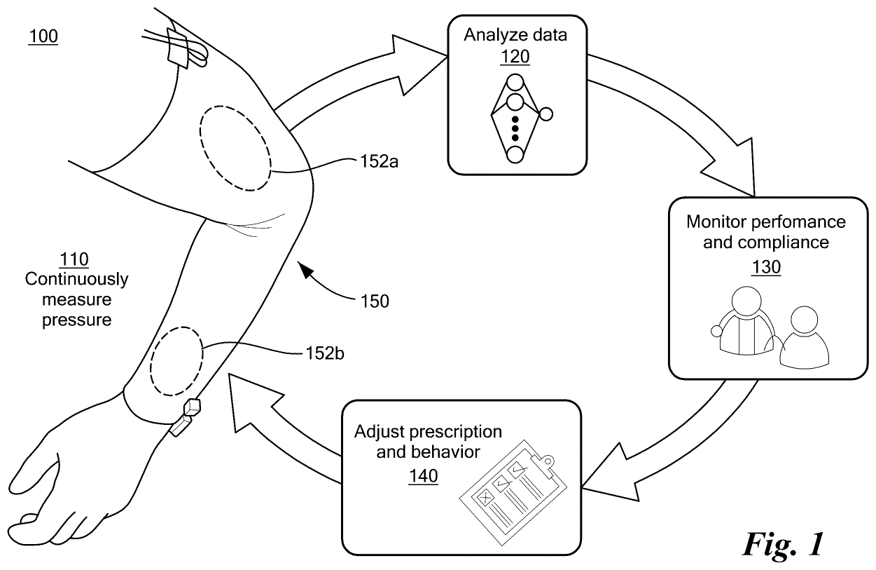

Referring now to FIG. 1, in process 100 a sensorized wearable garment 150 can be used to evaluate the performance of and improve a user's compliance with the wearable garment 150. The illustrative wearable garment 150 shown in FIG. 1 is a compression sleeve designed to fit over a user's arm and apply pressure thereto. The general concepts and structures disclosed herein can be used in conjunction with other types of wearable garments, such as garments worn on the leg or torso. For example, embodiments of the present disclosure may be integrated into vests, headbands, gloves, socks, etc. In some cases, a sensorized garment may be designed to be worn over an entire limb (e.g., whole leg or whole arm) or just a part of a limb (e.g., upper arm, lower arm, thigh, shin, etc.).

In a first step 110, the wearable garment 150, on a continuous or periodic basis, can collect data through one or more sensors 152a, 152b that are affixed to, embedded within, or otherwise integrated with the garment 150. Sensors 152a, 152b are operable to measure how much pressure the garment 150 is exerting on the user's arm (or other body part) at one or more positions/points there along. In the example shown, a first sensor 152a can be positioned near one end of the wearable garment 150 (e.g., near the user's wrist) while a second sensor 152b can be positioned near an opposing end (e.g., above the user's elbow).

In a second step 120, the collected data is analyzed. The collected data is first transmitted from the wearable garment 150 to a location for data analysis. The data may be transmitted through Bluetooth and/or through Wi-Fi. A Bluetooth transmitter or other wireless transmitter may be embedded into the wearable garment, enabling a wireless connection to the wearable garment. Alternatively, a wired connection may be used to transmit the data. The collected data may be transmitted to a number of different locations for further analysis. For instance, the data may be transmitted to a smartphone, tablet, desktop, a physical/virtual server (e.g., a server associated with a cloud-based computing environment), or other remote computing device. In some cases, the data may be transmitted to one or more applications running on such a remote computing device. In some cases, the data may be transmitted to a cloud-based storage system.

The collected data may be loaded into a data store (e.g., a local or cloud-based relational database or other type of data store) or provided to one or more applications for further analysis. In some cases, the collected data may be imported into an application hosted in a cloud computing environment, such as AMAZON AWS, GOOGLE CLOUD PLATFORM, MICROSOFT AZURE. In some cases, the data may be imported into an application for managing medical data, such as EPIC. In some cases, collected data may be stored by a microcontroller (e.g., using local file storage) to limit Wi-Fi usage and/or when Wi-Fi is not available. In some cases, collected data may be imported into a spreadsheet application, such as a GOOGLE SHEETS, configured to present a dashboard of pressure, usage duration, battery levels, device status, and various other metrics of interest.

In some embodiments, machine learning may be used to analyze collected data. For example, unsupervised learning (particularly clustering) may be used to learn a pressure threshold in real time that indicates whether or not the sleeve is being worn by a user (which can then be used to measure usage duration). If data is available from a population of users, a neural network, LSTM, transformer, etc. may be used to learn larger insights about the compression garments and their usage. These can be used to provide real-time feedback to patients directly, or doctors can use them to advance the state of the art of treatment for particular users or the general population.

The resulting data may be used to provide notifications to the user or another person, such as notifications related to usage of the garment by the user and/or performance of the wearable garment (e.g., whether the garment is exerting the pressure gradient on the user appropriate for treating a medical condition). The collected data may be displayed within a dashboard that gives real-time analysis of various metrics to the user. Those metrics may include pressure, usage duration, battery levels, device status, etc.

In a third step 130, the analyzed data can be monitored to determine if the wearable garment 150 is performing as intended (e.g., whether it is applying a prescribed amount of pressure to the user) and/or whether the user is complying with prescribed usage of the garment 150 (e.g., whether the user is wearing the garment at least X hours a day). In some embodiments, step 130 may include comparing analyzed data (from step 120) to one or more predetermined thresholds to determine if the garment is performing as intended and/or whether the user is complying with prescribed usage thereof. Such techniques are described in further detail below. In some embodiments, the analyzed data may be presented within or otherwise made accessible by one or more user computer applications, such as web application, a mobile application, a desktop application, etc. The user and/or their physician may utilize such applications to monitor the performance of and compliance with the wearable garment. In some embodiments, step 130 can include automatically generating and sending a notification (e.g., an email, text message, in-app notification, etc.) to a physician and/or user if it is determined that the garment 150 is not performing as intended and/or the user is not complying with the prescribed usage thereof.

In a fourth step 140, the wearable garment 150 and the treatment

prescribed for the user may be adjusted based on the results from step 130. For example, if it is determined that the garment 150 is not exerting the prescribed amount of pressure on the user, then a different garment type, style, size, etc. may be prescribed to the user. As another example, if it is determined that the user is not wearing the garment 150 at least X hours a day, where X is a number prescribed by a physician, then the physician may direct the user to adjust their behavior so as to comply with their prescription. As another example, if it is determined that the garment 150 is performing as expected and that the user is adhering to their prescription, but that the user's condition is not improving, then the prescription may be adjusted in one or more of the following ways: the amount of time per day the user wears the garment may be increased or decreased; the garment type, style, size, etc. may be changed; or the placement of the garment 150 on the user may be adjusted. Additional discussion about how a wearable garment can be prescribed to a user, and how such a prescription can be adjusted, is provided below.

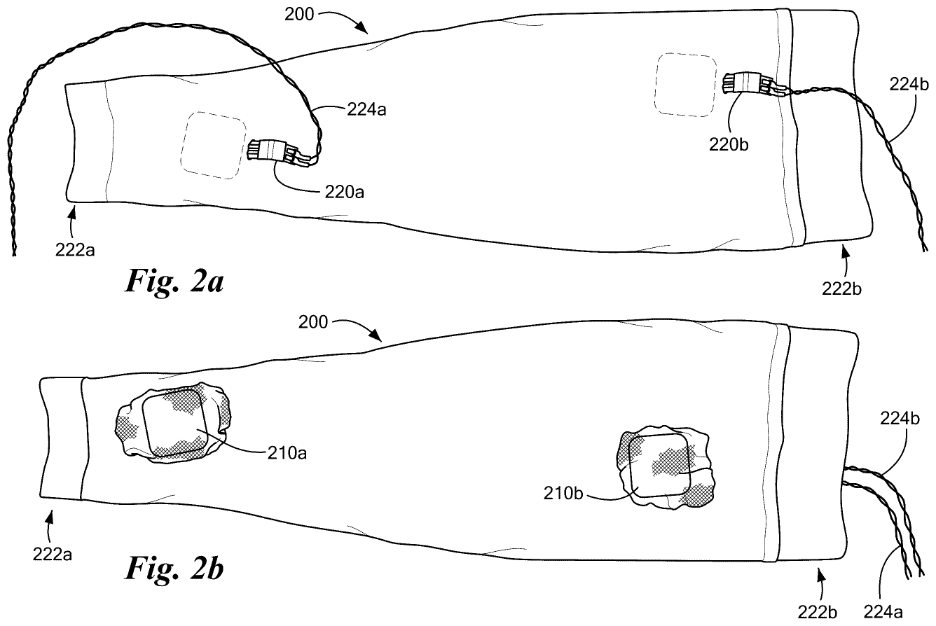

FIGS. 2a and 2b show an example of a wearable garment 200 formed of compression fabric and having one or more integrated resistive sensors, according to embodiments of the present disclosure. As illustrated in FIG. 2, two or more resistive sensors 210a, 210b (210 generally) may be affixed to an inside surface of the garment 200. The sensors 210a, 210b may be affixed to the garment 200 using glue or another adhesive. In some embodiments, the wearable garment 200 may include multiple layers of fabric and sensors 210 may be positioned between the multiple layers such that they are enclosed or embedded within the garment 200. Various other means of integrating sensors within compression fabric may be used in accordance with the present disclosure, including means described hereinbelow.

Sensors 210a, 210b may be electrically coupled to a microcontroller (not shown) via respective wires 224a, 224b. In more detail, a first sensor 210a may include a connector (or “port”) 220a to which a first wire 224a can connect, and a second sensor 210b may include another connector 220b to which a second wire 224 can connect. In some embodiments, two or more sensors may be wirelessly coupled to a single microcontroller. In other embodiments, different sensors may be connected to different microcontrollers. For example, multiple controllers, associated with and located near individual sensors, can be configured to communicate with each other wirelessly and thus eliminate the need for wires on the sleeve. Additional details regarding resistive sensors 210a, 210b and the microcontroller to which they are connected are provided below. As used here, the term “wire” can refer to one or more conductive paths. For example, a “wire” may comprise two conductors in a twisted pair arrangement.

As seen in FIG. 2b, first sensor 210a may be positioned towards a first end 222a of the wearable garment 200 (e.g. the end worn over a user's wrist) and second sensor 210b may be positioned towards a second, opposite end 222b of the wearable garment 200 (e.g. the end worn over the user's elbow). Other numbers and positions of sensors may be used. Moreover, while FIGS. 2a and 2b illustrate a sleeve-type wearable garment 200 configured to fit over a user's arm, in other embodiments, the wearable garment could be designed to fit on other body parts, such as a user's leg or a portion of the torso.

As previously discussed, wearable garment 200 may be made of a compression fabric. Compression fabric can provide a desired pressure gradient along a length of the wearable garment 200. For example, garment 200 may be about 36 cm long and designed to exert a graduated pressure between 20 mm Hg to 30 mm Hg along that length. For instance, a Class I sleeve may be used as standard compression to provide gradient pressure of 20 mm Hg to 30 mm Hg along the length of the user's arm. Additional ranges of gradient pressure may be used, such as from 10 mm Hg to 20 mm Hg and 30 mm Hg to 40 mm Hg. However, given that arm contours are not uniform amongst users, true gradient pressure dosing may be unknown. Following, if there are gradient interruptions, this may directly impact treatment outcome. Using the structures and techniques disclosed herein, appropriate pressure dosing can be ensured through the life of a garment, capturing decreased garment effectiveness with age, and need for replacement. Further, disclosed embodiments allow for real-time adherence monitoring and quantification of intervention dose, further defining the effect of true dose on BCRL outcomes. When used to make a sleeve for a user's arm, the compression fabric provides a gradient pressure which is tightest at the first end 222a (e.g. the end worn over a user's wrist) and decreases proximally toward the second end 222b (e.g. the end worn over the user's axilla or the elbow).

Resistive sensors 210a, 210b can be attached to the wearable garment 200 in a number of different ways, for example through stitching, epoxy resin, or glue. The attachment holds the resistive sensors 210a, 210b in place and enables the wearable garment to be soft, safe, and comfortable. Other attachment techniques may be used to allow the resistive sensors 210a, 210b to be removable, while still enabling the wearable garment to be comfortable for the user. The removability enables easier washing, repairs etc.

As previously discussed, resistive sensors 210a, 210b may be used to collect data about how much pressure the garment 200 is exerting on the user at different points in time (e.g., throughout a day). Certain placements of the resistive sensors 210a, 210b may be useful in ensuring the appropriate data is collected. For instance, in an embodiment with two resistive sensors 210a, 210b, such as those shown in FIGS. 2a and 2b, positioning the sensors on opposite ends 222a, 222b of the wearable garment 200 allows the resistive sensors 210a, 210b to measure a pressure gradient along the wearable garment 200. In alternative embodiments, two sensors be positioned on the same end of the wearable garment, e.g., both at end 222a or both at end 222b. In alternative embodiments, one sensor may be positioned on the wearable garment or there may be more than two sensors positioned on the wearable garment. The sensors may be positioned fully on one end of the wearable garment or spread along the length of the wearable garment.

In the embodiment of FIGS. 2a and 2b, the two resistive sensors 210a, 210b may be positioned on the same side of the wearable garment 200. The two resistive sensors 210a, 210b may be attached to opposite sides of the sleave or to the same side of the sleave (as shown in FIGS. 2a and 2b).

Disclosed embodiments can be used for medical treatment/evaluation, including but not limited to lymphedema treatment evaluation. In using one or more sensors, the accuracy and repeatability over time of the sensors must be sufficient to measure exerted pressure and usage duration for lymphedema treatment evaluation. Based on consultations with expert clinicians, it has been determined pressure should be measured to within approximately ±2 mmHg and usage duration should be estimated to within approximately fifteen minutes over a twelve hour period. Performance should also be reliable in varying environmental conditions such as ambient temperature or pressure, and despite operating at a dynamic interface between the human arm and the stretchable sleeve. Users are fitted for and instructed to wear the wearable garment for greater than or equal to twelve hours per day.

Disclosed sensor embodiments are robust enough for daily wear including common activities and may be unobtrusive such that they do not cause the user any discomfort or adverse effects. Further, a scalable and customizable fabrication approach streamlines sleeve integration to allow for widespread deployments. An important goal for the deployable wearable system is to maintain medically relevant accuracy while reducing how much calibration is required. This is coupled with reducing sensitivity to ambient conditions; for example, if the wearable garment is fitted in an air-conditioned doctor's office at sea level, and then the user travels to an elevated city on a hot day, the sensor would ideally continue to generate actionable data without requiring the user to calibrate. This goal can also help inform sensor design parameters, as discussed further below.

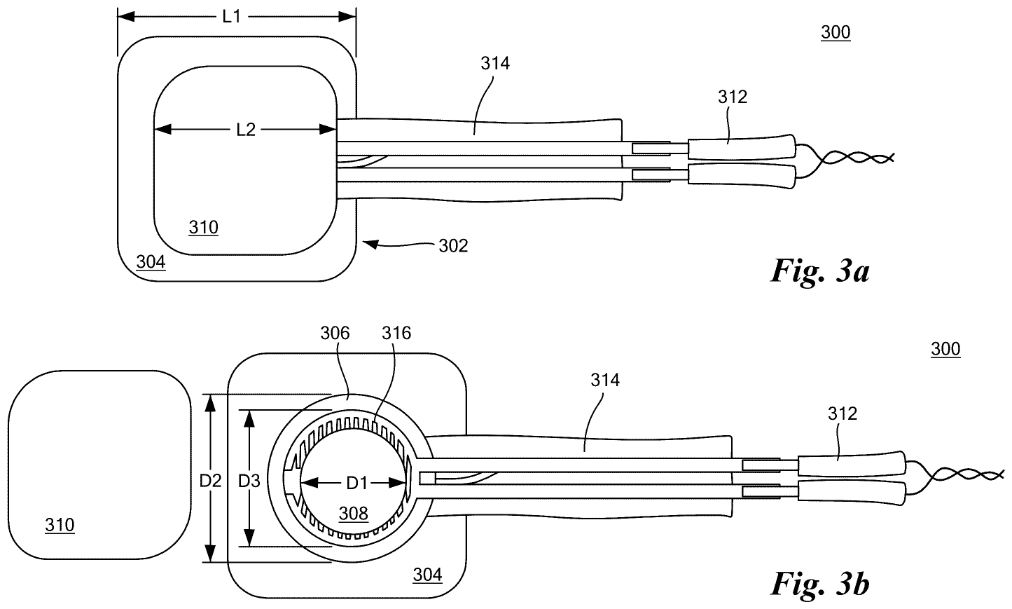

FIGS. 3a and 3b show an example of a resistive sensor 300, according to some embodiments. Resistive sensor 300 may be the same as or similar to either resistive sensor 210a, 210b described above in conjunction with FIGS. 2a and 2b.

Illustrative resistive sensor 300 includes a transducer portion 302 having a backing layer 304, a transducer 306, a middle layer 308, and a top layer 310. Transducer 306 includes an active area 316 that changes in electrical resistance in response to mechanical pressure or force (e.g., as a result of compression forces from a wearable garment). For example, active area 316 may include a series of conductive traces formed on a flexible substrate. The sensor 300 further includes a connector 312 electrically coupled to the active area 316 via conductive leads 314. Via connector 312, resistive sensor 300 can be connected to a voltage divider to yield a voltage that depends on applied pressure. In some cases, the voltage divider may be part of a microcontroller.

As seen in FIG. 3a, top layer 310 can be assembled over backing layer 304 to provide a sheath around transducer 306. Thus, the illustrative sensor 300 may be described as being a “sheathed” sensor. Backing layer 304 may have a generally square shape, with edge length L1, to avoid sinking into the user's arm. Top layer 310 may also have a generally square shape, with edge length L2. In one example, L1 may be about 2.5 cm and L2 may be about 2.0 cm. The corners of backing layer 304 and/or top layer 310 may be rounded to increase user comfort.

Backing layer 304 and top layer 310 may be formed out of a flexible plastic material such as polyethylene terephthalate, high-density polyethylene, low-density polyethylene, polyvinyl chloride, polypropylene, polystyrene, or polycarbonate. For example, flexible polystyrene sheets with a thickness of 0.5 mm may be used to form backing layer 304 and top layer 310.

As shown, middle layer 308 may be positioned over a center of active area 316 to prevent top layer 310 from coming into direct contact therewith, thereby causing pressure to be distributed across the active area 316. Middle layer 308 may be made of the same or different material as layers 304, 310. For example, in some cases, middle layer 308 may be more rigid than layers 304, 310. Middle layer 308 can have a substantially circular shape with diameter D1. In one example, D1 may be about 1.1 cm.

Transducer 306 may be affixed to backing layer 304 using glue or another adhesive. Middle layer 308 may adhered on one side of transducer 306 (e.g., using tape) to prevent movement while not applying pressure. Finally, top layer 310 may be positioned over transducer 306 and middle layer 308 and affixed using a mechanical fastener, adhesive, or other means.

Transducer 306 can have a circular shape with diameter D2 and its active area can have a circular shape with diameter D3, as shown in FIG. 3b. In one example, D2 can be about 1.8 cm and D3 can be about 1.3 cm. In some cases, transducer 306 can have a thickness of about 0.6 mm. While having a thin profile and small surface area allows the transducer 306 to be unobtrusive, it also allows it to sink into indentations of the skin and potentially yield inaccurate results. Furthermore, uniformly stretching the sleeve over the transducer 306 may cause its inactive edge to support much of the pressure and preclude successful measurements. To address these challenges, the resistive sensor 300 sheathed by layers 304, 308, 310, as previously discussed. The resulting resistive sensor 300 provides a larger surface area for the sleeve interface and focuses/guides exerted pressure onto the resistive sensor's active area.

In an embodiment, the transducer 306 may be the circular Force Sensing Resistor (FSR) from INTERLINK ELECTRONICS. Transducer 306 may have a nominal sensitivity range of 0.1-10.0 N (5.7-565 mmHg if force is distributed over the entire active area).

The inventors have demonstrated a resistive sensor similar to that shown in FIGS. 3a and 3b can be used to collect data and yield accurate results related to the performance of, and compliance with, wearable garment treatment. The resistive sensor's 300 collects data and is expected to exhibit an exponential mapping between applied pressure and resistance. The collected data from resistive sensor 300 can be analyzed using one or more statistical techniques to determine its accuracy, as described next. Pressure can be substituted for force since the constant factor of area can be encapsulated within the optimized a and b:

$${R}_{sensor} = ( a ) {( Pressure )}^{( - b )} \qquad ( 1 )$$

The resulting resistance is then converted to voltage via a voltage divider with a fixed resistor. The sensed voltage at their junction is given by:

$${V}_{sensor} = ( {V}_{regulated} ) ( \frac{{R}_{divider}}{{R}_{divider} + {R}_{sensor}} ) \qquad ( 2 )$$

where R_{divider} is selected to maximize the range of measured voltages around the expected operating point. Currently, a 5.6 kΩ resistor is used. Combining Equations 1 and 2 yields:

$$Pressure = {[ \frac{( a ) ( \begin{matrix}{V}_{sensor} \\ {V}_{regulated}\end{matrix} )}{{R}_{divider} ( 1 - \frac{{V}_{sensor}}{{V}_{regulated}} )} ]}^{( \frac{1}{b} )} \qquad ( 3 )$$

This equation provides a well-founded curve to approximate the sensor response while only using two tunable parameters.

Fitting to each individual resistive sensor yields an average error of 0.5±0.2 mmHg (2.0%±0.7%), fitting to a single previous sensor without calibration yields an average error of 7.8±5.0 mmHg (28.2%±19.5%), and fitting to all previous sensors yields an average error of 5.4±3.5 mmHg (19.8%±15.2%). To adjust a curve using the single-point calibration paradigm, the analysis explored computing a new a or b as well as adding an offset to shift the curve. Adding an offset was found to be most accurate and yielded an average error of 0.7±0.2 mmHg (2.6%±0.7%).

Results suggest that using FSRs without calibration may not be sufficiently accurate for the current application. Variability may be due to the sensors, or to factors such as sensitivity to placement. Using a single-point calibration yields accuracies that are comparable to a pneumatic sensor, such as those pneumatic sensors discussed below. However, the resistive sensor 300 may need semi-rigid sheathing, such as the backing layer 304, middle layer 308, and top layer 310, to appropriately distribute pressure onto the resistive trace 370, which introduces edges on the person's skin under high pressures. They also exhibit higher sensitivity to ambient temperature, and characterization results suggest that at least a single-point calibration routine is required to obtain sufficient accuracy.

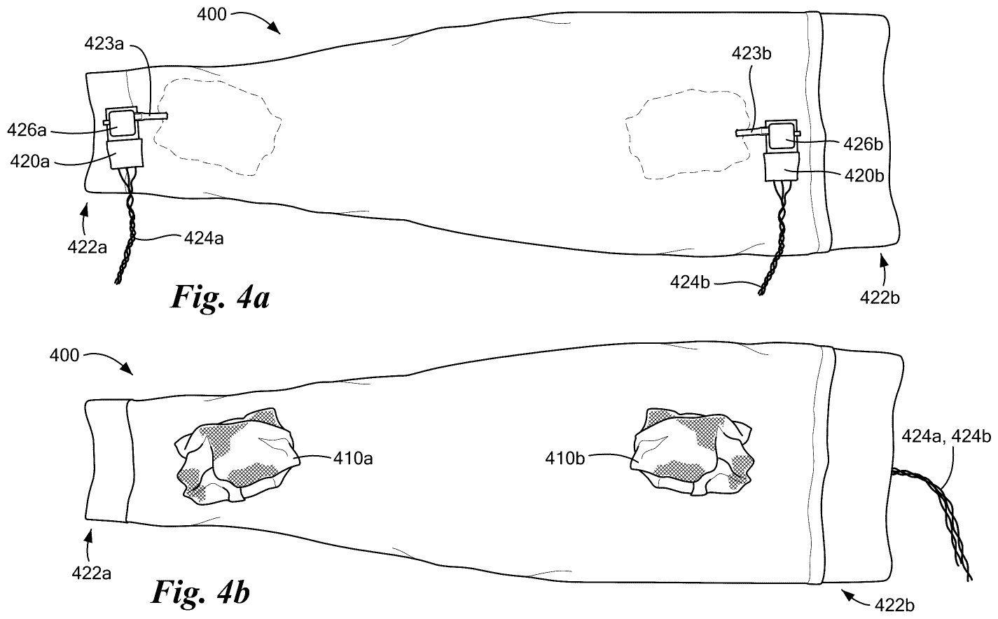

FIGS. 4a and 4b show a wearable garment 400 having pneumatic sensors, according to some embodiments of the present disclosure. Similar to the garment described above in the context of FIGS. 2a and 2b, wearable garment 400 may be formed of a compression fabric and designed to be worn over a user's arm, leg, torso, or other body part. In one example, garment 400 may have a length of about 36 cm.

In the example of FIGS. 4a and 4B, two pneumatic sensors (sometimes referred to as “bladder-type” sensors or simply “bladder” sensors) are provided, one located toward a first end 422a of the garment (e.g., the end worn over a user's wrist) and another located toward a second, opposite end 422b of the garment (e.g. the end worn over a user's elbow). The first pneumatic sensor comprises a first bladder 410a connected to a first transducer 426a via a first tube 423a (e.g., a flexible tube or air hose), and the second pneumatic sensor comprises a second bladder 410b connected to a second transducer 426b via a second tube 423b. The bladders 410a, 410b may be filled with air, CO₂, or another gas or fluid; the corresponding tubes 423a, 423b may sealed at both ends to provide an air tight connections between bladders 410a, 410b and respective transducers 426a, 426b.

As shown in FIG. 4b, bladders 410a, 410b may be affixed to an inside surface of the garment 400 using tape, glue, epoxy, or another adhesive. In other embodiments, bladders 410a, 410b may be embedded within garment 400. For example, garment 400 may be provided as having two layers of fabric and bladders 410a, 410b may be positioned therebetween. As shown in FIG. 4a, transducers 426a, 426b may be positioned on an outside surface of garment 400 and tubes 423a, 423b may extend through garment (e.g., via through holes cut therein) to connect to the bladders 410a, 410b on the inside surface. In some embodiments, transducers 426a, 426b may be affixed to the garment 400 using adhesive or a mechanical fastener. Various other approaches may be used to integrate pneumatic sensors into a wearable garment.

Transducers 426a, 426b may be operable to generate, as output, electrical signals that have a voltage responsive to the fluid pressure within sealed bladders 410a, 410b and connecting tubes 423a, 423b. As one example, transducers 426a, 426b may include be provided as D2-P4V-Mini pressure transducers from ALL SENSORS. The pressure transducer converts the pressure acting on it into electrical signals that are relayed to the controller. In some embodiments, a transducer 426a, 426b may have an operating pressure range of ±30 inches of water (±56 mmHg) and may output an analog voltage proportional to the difference in pressure between two input ports. The pouch tubing 423a, 423b may be connected to the positive port, and the negative port can be left open or sealed. The pneumatic sensors may be connected to a microcontroller (not shown) configured to process the voltage signals generated by transducers 426a, 426b to determine pressure exerted by the garment 400 at different points along its length and, thus, at different positions on the user's body. In more detail, sensors can include connectors 420a, 420b into ends of respective wires 424a, 424b may be connected, with the other ends of wires 424b, 424b being connected to a microcontroller. In other embodiments, pneumatic sensors may be wirelessly connected to the microcontroller.

A skilled artisan will appreciate that the wearable garment embodiment shown in FIGS. 4a and 4b may have a design similar to that described above in conjunction with FIGS. 2a and 2b. For example, wearable garments may be constructed using similar fabrics, having similar numbers and placements of sensors. Moreover, like resistive-sensorized garment of FIGS. 2a and 2b, the pneumatic sensorized garment of FIGS. 4a and 4B can be used for medical treatment/evaluation, including but not limited to lymphedema treatment evaluation.

While FIGS. 4a and 4b illustrate a sleeve-type wearable garment 400 configured to fit over a user's arm, in other embodiments, the wearable garment could be designed to fit on other body parts, such as a user's leg, a portion of the torso, head, hand, or foot. For example, the wearable garment could be a vest, headband, glove, or sock. The embodiment in FIGS. 4a and 4b show the wearable garment 400 as a compression sleeve, wherein the pneumatic sensors are capable of measuring when a user is and is not wearing the wearable garment 400, how much pressure wearable garment 400 exerts against the skin at various points, such as near the ends 422a, 422b of garment 400.

As shown in FIGS. 4a and 4b, pneumatic sensors can be integrated into wearable garment 400. For example, bladders 410a, 410b can be stitched into the garment or adhered using epoxy or glue. In some cases, the sensors may be attached to the garment in a removable manner.

One or more pneumatic sensors may be provided to collect data on the user. Certain placements of the pneumatic sensors bladders 410a, 410b may be useful in ensuring the appropriate data is collected. The pneumatic sensor bladders 410a, 410b may be arranged along the wearable garment 400 such that the one or more sensors can measure how much pressure the compression fabric is exerting against the skin of the user at various points along the arm. For instance, in an embodiment with two pneumatic sensors, such as shown in FIGS. 4a and 4b, the positioning on each end 422a, 422b of the wearable garment 400 allows the pneumatic sensors to measure a pressure gradient along the wearable garment 400. Additionally, the two pneumatic sensors may be positioned on the same side of the wearable garment 400. The two pneumatic sensors may be positioned such that they are both on the inside or the outside of the user's arm. Further, one pneumatic sensor may be positioned at one end of the wearable garment 400 and the other pneumatic sensor at another end of the wearable garment 400. As discussed above, in alternative embodiments, different placement of sensors is possible.

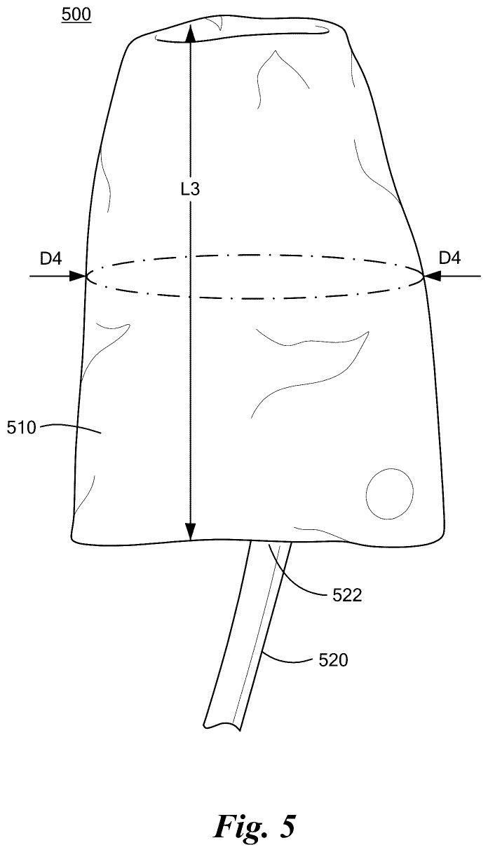

FIG. 5 shows a portion of a pneumatic sensor 500 that may be integrated within a wearable garment, according to some embodiments. The illustrative pneumatic sensor 500 has a bladder 510 and a flexible tube 520 attached thereto. Bladder 510 may be the same as or similar to a bladder 410a, 410b of FIG. 4b and flexible tube 520 may be the same as or similar to tubes 423a, 423b of FIG. 4a. Of note, FIG. 5 does not show the transducer or electrical connector, which may also be considered to form part of pneumatic sensor 500.

Tube 520 can have a first end removably or permanent connected to the bladder 510 and another end connected to a transducer (not shown). In some embodiments, bladder 510 may include a port (e.g., at or near the location indicated 522 in FIG. 5) via which tube 520 can be connected and disconnected multiple times. In other embodiments, tube 520 can be permanently adhered to bladder 510 using glue, epoxy, etc.

Bladder 510 can have an active length L3 and a diameter D4, as shown. The diameter D4 may correspond to the diameter of the bladder 510 when inflated. In some cases, L3 may be in the range of 1 cm to 6 cm, for example 4 cm. In one example, L3 may be about 6 cm and D4 may be about 0.75 cm. In some cases, bladder 510 may have a thickness of about 1 mm thick or less. It should be understood that these dimensions are merely illustrative and that smaller or larger dimensions may be used.

Bladder 510 can be filled with a gas, such as air or CO₂, or another fluid in sufficient amount to create a bulge under the surface of the wearable garment of approximately 4 mm; this helps unify how the wearable garment exerts pressure on each sensor and standardizes the user experience. The volume of gas within bladder 510 may be in the range of 1 mL to 7 mL, depending, for example, on its active length L3 and a diameter D4.

Bladder 510 and tube 520 may be made be made out of a flexible plastic material such as polyethylene terephthalate, high-density polyethylene, low-density polyethylene, polyvinyl chloride, polypropylene, polystyrene, or polycarbonate. In some embodiments, bladder 510 may be formed out of a repurposed vinyl powder-free medical examination glove, such a glove made by MEDPRIDE. In some embodiments, tube 520 may be made out of a flexible silicone tubing with a 1.5 mm inner diameter, a 3 mm outer diameter, and a durometer of 50A is inserted approximately 1 cm into the pouch. In some cases, tube 520 may have a length of about 4 cm. The interface 522 between the bladder 510 and the tube 520 may be sealed using Smooth-On Sil-Poxy.

The pneumatic sensor 500 uses a simple fabrication method and commodity materials to yield a robust pressure sensor with linear response. The softness is important for a user's comfort and safety, especially in high-pressure and long-duration applications. It can also be rapidly prototyped and customized. Key design considerations include the pouch size, how much it is inflated, and whether to use an open or closed reference port. A smaller and less inflated pouch may be more desirable since it minimizes obtrusiveness for the person wearing the sleeve. However, since the user's arm is deformable, a pouch that is too small may sink into an indentation of the skin and yield inaccurate pressure estimations of the sleeve passing on top of it. Under-inflating the pouch may also allow it to be flattened by the sleeve, blocking the inlet of the tube inside the pouch. Importantly, the bladder pouch is also soft and thus well-suited to wearable applications. Further, the transducer or tubing must be mounted on the sleeve which may slightly increase obtrusiveness, and the transducer requires an outlet to the atmosphere which may complicate encapsulation for protection and waterproofing.

The collected data from pneumatic sensor 500 can be analyzed using one or more statistical techniques to determine its accuracy using, as described next.

Voltage is measured with respect to pressure:

$$Pressure = ( \frac{{V}_{sensor}}{{V}_{regulated}} ) m + b \qquad ( 4 )$$

Where m and b are parameters optimized via the curve_fit function of the SciPy Python package. This is first done for each sensor individually. The average error of these five fits was 0.8±0.2 mmHg (2.9%±0.9%) across all tested pressures.

To increase accuracy further, since characterization errors will compound with errors induced by ambient conditions, a minimal calibration procedure is used that assumes a user has their sleeve measured once at a clinical visit. The current implements this paradigm include fitting a curve to all sensors except one, then adjusting the curve for the left-out sensor based on a single calibration point. It simulates cases where the calibration measurement is 15 different pressures evenly spaced between 16 and 44 mmHg, to account for differing wearable garment fits across different users.

For each case, the program computes a new b using Equation 4 and an m optimized to all other sensors. It then evaluates the adjusted curve at pressures within ±6 mmHg of the calibrated pressure, since the pressure of a deployed sleeve is expected to remain relatively consistent for a given subject between visits to the doctor's office. Using this single-point calibration paradigm, the expected error was 0.5±0.1 mmHg (1.8%±0.3%). Through the calibration and the collection of pressure data, it can be determined whether or not the sleeve is being worn by the user. Further, the data from each sensor can be compared to determine the pressure gradient across the sleeve. By determining when and if the user is actively wearing the sleeve and the pressure gradient across the sleeve, the prescribed treatment for the user can be optimized.

Through the calibration and the collection of pressure data, it can be determined whether or not the wearable garment is actively on the user. Further, the data from each pneumatic sensor 500 can be compared to determine the pressure gradient across the user. By determining when and if the user is actively wearing the sleeve and the pressure gradient across the wearable garment, the prescribed treatment for the user can be optimized.

While FIGS. 2a, 2b, 4a, 4b shows examples of wearable garments with two particular types of sensors (i.e., resistive and pneumatic sensors), a skilled artisan will appreciate that various other types of sensors may be utilized in accordance with the general structures and techniques sought to be protected herein. In an embodiment, sensors comprising piezoresistive materials and sensors comprising capacitive sensors may be used. Piezoresistive materials decrease electrical resistance when pressure is applied.

In an embodiment, one or more sensors may be integrated into a wearable garment through embroidery or knitting. For example, conductive threads can be used as a method to create sensors and for integrating them into the garment. These techniques can be used produce sensors for integrated into a garment similar to the those shown herein, or they could be used in the process of creating the garment itself so the sensing is more integrated with the compression material rather than being an add-on. Embroidery and knitting can also allow digital fabrication. For example, computational design tools may be used to rapidly customize the shape and size and coverage of the sensors on the sleeve and/or personalize it to a user.

Other potential sensors may include: force sensors, contact sensors, or motion sensors. Contact sensors detect when the sleeve is worn by the user. This is done through one or more electrodes (to detect electrical conductance of the skin/arm). Alternatively, contact sensors may include mechanical sensors or thresholder force sensors. Motion sensors such as an accelerometer, gyroscope, or combined Inertial Measurement Unit (IMU) sensor may be used. The motion sensors could be used to detect when the sleeve is worn by the user, estimate the user's pose/position, monitor the user's activity levels, or detect specific activities.

In some embodiments, additional sensors could be incorporated to collect general health and activity-monitoring data. These sensors may include: muscle activity sensors, heart rate sensors, skin conductance sensors, sweat detection sensors, temperature sensors, blood pressure sensors, light sensors, or sensors to measure the strain of the sleeve material. Skin conductance sensors may include galvanic skin response (GSR) sensors, which collect data related to stress or sudden events. Temperature sensors may collect data on the temperature of the skin or the ambient environment. All sensors are configured to withstand repeated donning, doffing and removal for handwashing of the compression fabric without affecting sensor integrity.

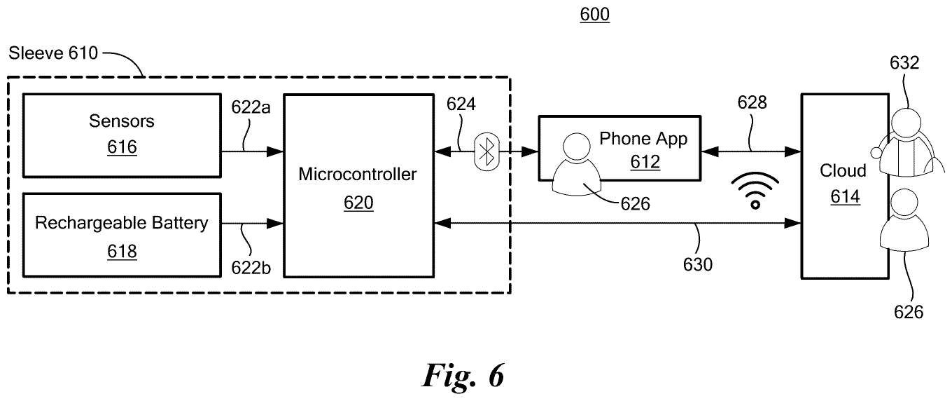

FIG. 6 depicts a system 600 for monitoring performance of and enforcing compliance with a wearable garment, according to some embodiments. A sensorized garment 610 (e.g., a sleeve) can include one or more sensors 616, a battery 618, and a microcontroller 620. Sensors 616 can include pressure sensors similar to those described above in the context of FIGS. 2a, 2b, 3a, and 3b or pneumatic sensors similar to those described above in the context of FIGS. 4a, 4b and 5.

Sensors 616 can be connected to microcontroller 620 one or more first signal paths (e.g., wires) 622a. Battery 618 can be connected to microcontroller 620 to provide power thereto, as indicated by line 622b. In some cases, battery 618 may be a rechargeable battery. In other cases, battery 618 may be user-replaceable.

Microcontroller 620 can collect data from sensors 616 and transmit that data to one or more external processors/computers for analysis and monitoring. For example, as shown in FIG. 6, microcontroller 620 can transmit data to a smartphone application 612 via a wireless link 624 (e.g., a Bluetooth connection).

As another example, microcontroller 620 can transmit data to a cloud application 614 (i.e., an application hosted within a cloud computing environment) via a wired over computer network 630. In some cases, the smartphone application 612 may alternatively or additional provide sensor data to cloud application 614, via computer network 628. Computer network 628 and/or 630 may include a wired network, wireless network (e.g., Wi-Fi), or a combination thereof. Microcontroller 620 can transmit data to smartphone application 612 and/or cloud application 614 in a secure manner (e.g., using an encrypted communications link) and on a continuous or periodic basis. Microcontroller 620 can include one or more radios for Bluetooth, Wi-Fi, or other forms of wireless communication.

Smartphone application 612 may enable a user 626 access data collected from the sensorized garment 610. Cloud application 614, may enable the user 626 and/or other users 632 to access data collected from garment 610. The other users 632 may include doctors, nurses, medical guardians, or other persons authorized to access such data on behalf of user 626. Cloud application 614 may include security-compliant software for managing data collected from garment 610.

As shown in FIG. 6, microcontroller 620 and battery 618 may be integrated into garment 610. That is, the sleeve may be a self-contained apparatus. In other embodiments, microcontroller 620 and/or battery 618 may be physically separate from the garment 610. In this case, a portable carrier may be provided to the user for housing the microcontroller 620 and/or battery 618. The carrier may be designed to be worn around the user's waist or otherwise fastened to the user's body/clothing. In some cases, a Velcro strap may be provided for fastening the portable carrier to the user. In general, the portable carrier may be designed for both protection of the electronics and for aesthetics.

Microcontroller may be provided as a commercially available control board such as an ADAFRUIT QT PY ESP32-C3 microcontroller, which features Wi-Fi and Bluetooth. In some cases (e.g., to improve accuracy), an ADS1115 ADC may be provided to measure the sensor outputs and regulate voltage. In some cases, battery 618 may be provided as a 500 mAh single-cell lithium-ion battery to power microcontroller 620 and potentially other electronics. These components, along with a charging circuit and FSR voltage dividers, can be mounted on a 3 cm×4 cm breadboard. In the case of pneumatic sensors, P4V pressure transducers can be mounted on the sleeve near each pouch.

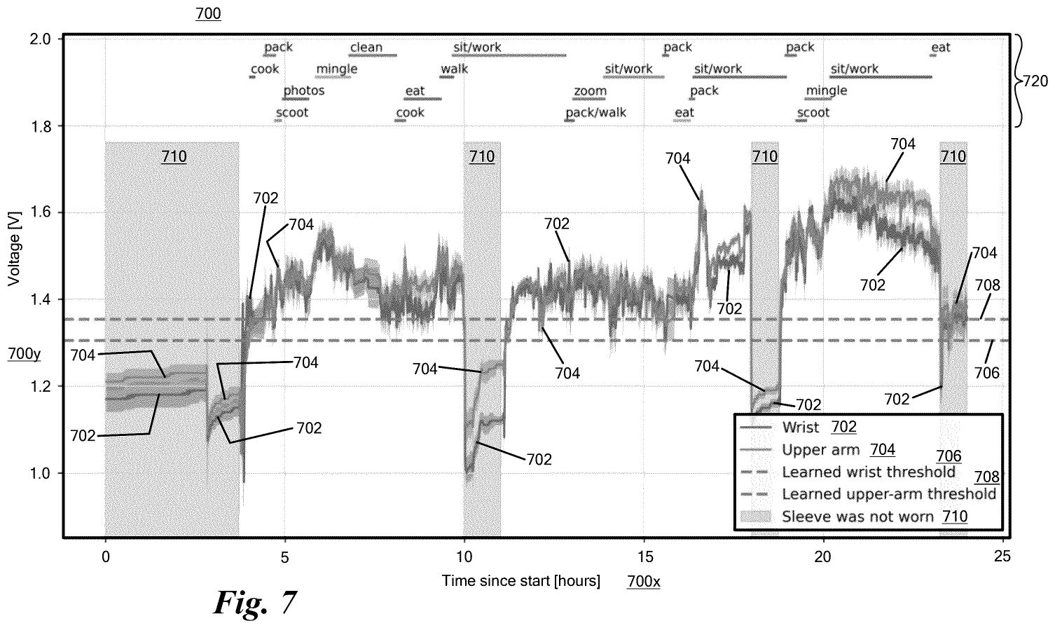

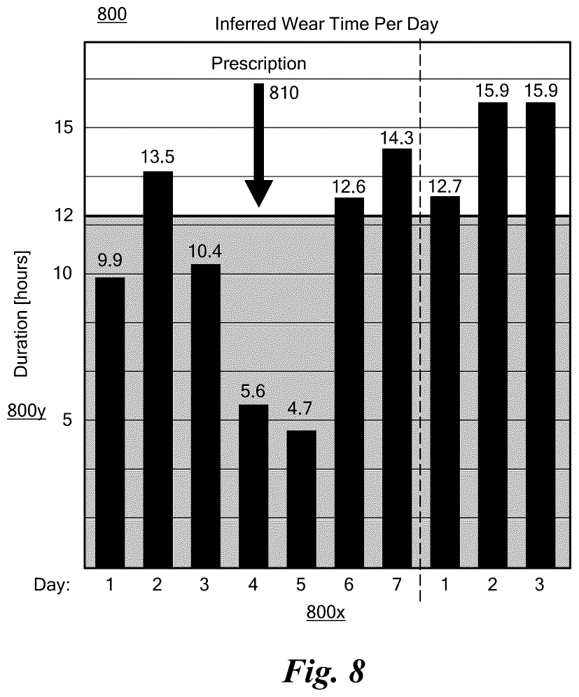

Microcontroller 620 can sample each sensor at a given rate, such as 1 Hz. On a periodic basis (e.g., every minute), microcontroller 620 can compute the mean and standard deviation of the ADC values. It can wirelessly transmit these calculated values to smartphone application 612 and/or cloud application 614. In some cases, cloud application 614 may correspond to a cloud-based spreadsheet application such as GOOGLE SHEETS, and collected/computed data may be automatically populated thereinto to provide a real-time dashboard (i.e., display) provides pressure and usage information. The data can provide insights about the wearable garment by continuously monitoring pressure. In some cases, and as discussed below in conjunction with FIGS. 7 and 8, one or more threshold values may be used to infer when the sleeve is worn and how closely the user is adhering to usage guidelines.

FIGS. 7 and 8 illustrate how data obtained from a sensorized wearable garment can be used to detect performance thereof and compliance therewith.

Referring to FIG. 7, in the example shown, a wearable garment with two sensors was worn by a user for multiple days, with one sensor located near the user's wrist and another sensor located on the user's upper arm. Graph 700 shows data collected from the wrist sensor 702 and data collected from the upper-arm sensor 704, over time. In more detail, horizontal axis 700xcorresponds to time (e.g., hours) and vertical axis 700y corresponds to sensor voltage readings. The shaded regions 710 correspond to times when the garment was not being worn. Other regions correspond to times when the garment was being worn by the user. In this example, data was collected while the user was performing various activities, such as cooking, cleaning, walking, etc. A description of these activities and the times at which they were performed is indicated by horizontal lines 720 near to top of the graph 700.

As can be seen in FIG. 7, both sensors consistently generate comparatively low, but non-zero, voltages when the user is not wearing the garment (i.e., in the regions 710). Thus, based on analysis data collected from the sensors, one or more sensor thresholds 706, 708 can be established and used to automatically detect when the user is wearing the garment. Sensor readings above a threshold can indicate that the user is wearing the garment at that time. As illustrated by graph 700, sensor thresholds 706, 708 can be used to accurately determine if the user is wearing the garment regardless of which activity the user is performing.

In some embodiments, different thresholds can be determined for different sensors. For example, as shown in FIG. 7, a first sensor threshold 706 may be established for the wrist sensor and a second sensor threshold 708 can be established for the upper-arm sensor. In this particular example, first sensor threshold 706 may be about 1.30 V and second sensor threshold 708 m may be about 1.35 V. The actual sensor threshold values can vary depending on the types of sensors used, the dimensions and configuration of the sensors, the type of sleeve/garment used, the position of the sensors on the garment, among other factors. In some cases, sensor thresholds may be established on a per-user and/or per-garment basis. In some cases, a sensor threshold may be dynamically adjusted over the course of a day. For example, a sensor threshold may be decreased during hours when the user typically sleeps. In some cases, sensor thresholds 706, 708 may be automatically determined (or “learned”) based on analysis of past sensor data collected for a particular user/garment. In some cases, sensor thresholds 706, 708 may be adjusted by a user/physician (e.g., via a smartphone or cloud application).

By comparing collected data to sensor thresholds 706, 708, an accurate measurement can be made of how long the user wears a garment over a given period (e.g., how many hours per day). This duration use can be compared to another threshold (“duration of use” threshold) to determine if the user is complying with the prescribed usage of the garment.

Referring to FIG. 8, an illustrative graph 800 has a horizontal axis 800x corresponding to days (with 10 consecutive days shown in this example) and a vertical axis 800y corresponding to duration (e.g., the number of hours the user was detected has having worn the garment in a given day). Graph 800 further includes a prescribed duration of use threshold 810 that can be set by the user's physician, for example. In the example of FIG. 8, the user complied with their prescription on 60% of the days for which data was collected.

According to some embodiments, if the measured duration of use in a given day, or over N consecutive days, is less than the prescribed threshold 810, a notification may be automatically generated and sent to the user and/or their physician. Such a notification can take the form of an email, text message, in-app notification, etc.

The processing and techniques described above in conjunction with FIGS. 7 and 8 may be implemented within and executed by system 600 of FIG. 6. More particular, said processing can be implemented/executed by smartphone application 612 and/or cloud application 614.

According to some embodiments, data collected from a sensorized garment can be used to monitor pressure to determine whether the sleeve is operating correctly. Such data can be analyzed to determine whether a target pressure (e.g., as prescribed by a physician) is achieved, and it also allows one to see pressure change over time-something doctors cannot do without the continuous monitoring provided by embodiments of the present disclosure. In more detail, under current treatment paradigms, physicians may measure pressure only during clinic visits and, thus, they only see controlled snapshots of pressure. The structures and techniques described herein can be used to give them much more data throughout all of a patient's activities. That is, embodiments of the present disclosure can provide physicians with additional data and additional tools to provide a higher level of care and to perform better research about treatment parameters.

In addition, data collected from a sensorized garment can be used to infer usage duration from that data as one important metric. It may be also be used to infer other information from the pressure, such as whether the patient's arm has started to swell or started to reduce its swelling, the garment fit, how the patient uses the garment, or whether the garment itself is operating correctly. Metrics about the patient's usage of the garment could include, for example, how long they wear it each day, how long they wear it for each usage period, whether they are using it correctly, whether they are following usage guidelines/prescriptions, etc. Metrics about the patient's condition/behavior could include. for example, monitoring their arm swelling and determining whether arm swelling has increased or decreased (or similar for other limbs/body locations), or other metrics relevant to sports, compression therapy, etc.

In some embodiments, one or more of these metrics may be used to provide (e.g., displayed by a computer application) feedback or insights to a physician, patient, supervisors, etc. Such feedback/insights that can be provided may include, for example, real-time reminders to wear the garment or about how to use the garment, summary reminders to wear the garment more or less often or to use it according to certain guidelines/prescriptions, reports about the garment's condition, reports summarizing the data and/or extracted insights, statistical analyses and reports, general analysis of the data, machine learning results based on the data, etc. In some embodiments, data may be processed from each garment/patient individually to generate personalized results, or aggregated from multiple garments/patients to create larger datasets and results that apply to a broader population.

Although reference is made herein to particular materials, it is appreciated that other materials having similar functional and/or structural properties may be substituted where appropriate, and that a person having ordinary skill in the art would understand how to select such materials and incorporate them into embodiments of the concepts, techniques, and structures set forth herein without deviating from the scope of those teachings.

Various embodiments of the concepts, systems, devices, structures and techniques sought to be protected are described herein with reference to the related drawings. Alternative embodiments can be devised without departing from the scope of the concepts, systems, devices, structures and techniques described herein. It is noted that various connections and positional relationships (e.g., over, below, adjacent, etc.) are set forth between elements in the following description and in the drawings. These connections and/or positional relationships, unless specified otherwise, can be direct or indirect, and the described concepts, systems, devices, structures and techniques are not intended to be limiting in this respect.

As used herein, the terms “comprises,” “comprising, “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean serving as an example, instance, or illustration. Any embodiment or design described herein as “exemplary” is not necessarily to be construed as preferred or advantageous over other embodiments or designs. The terms “one or more” and “one or more” are understood to include any integer number greater than or equal to one, i.e. one, two, three, four, etc. The term “connection” can include an indirect “connection” and a direct “connection.”

References in the specification to “one embodiment, “an embodiment,” “an example embodiment,” etc., indicate that the embodiment described can include a particular feature, structure, or characteristic, but every embodiment can include the particular feature, structure, or characteristic. Moreover, such phrases do not necessarily refer to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

For purposes of the description hereinafter, the terms “upper,” “lower,” “right,” “left,” “vertical,” “horizontal,” “top,” “bottom,” and derivatives thereof shall relate to the described structures and methods, as oriented in the drawing figures.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Subject matter described herein can be implemented in digital electronic circuitry, or in computer software, firmware, or hardware, including the structural means disclosed herein and structural equivalents thereof, or in combinations of them. The subject matter described herein can be implemented as one or more computer program products, such as one or more computer programs tangibly embodied in an information carrier (e.g., in a machine-readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or another unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this disclosure, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processor of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of nonvolatile memory, including by ways of example semiconductor memory devices, such as EPROM, EEPROM, flash memory device, or magnetic disks. The processor and the memory can be supplemented by, or incorporated in, special purpose logic circuitry.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out the several purposes of the disclosed subject matter. Therefore, the claims should be regarded as including such equivalent constructions insofar as they do not depart from the spirit and scope of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

What is claimed is:

1. A system comprising:
one or more sensors integrated within a wearable garment, the sensors configured to generate data responsive to pressure exerted by the wearable garment on a user; and
one or more processors coupled to the one or more sensors and configured to:
process the generated data to determine a duration of use of the wearable garment by the user; and
in response to determining that the duration of use is less than a predetermined duration of use threshold, generating one or more notifications.

2. The system of claim 1, wherein the duration of use threshold is prescribed by a physician.

3. The system of claim 2, wherein the wearable garment is a garment prescribed to the user for treatment of cancer-related lymphedema (BCRL).

4. The system of claim 1, wherein the wearable garment comprises compression fabric.

5. The system of claim 4, wherein the compression fabric provides a pressure of between 10 mm Hg to 40 mm Hg.

6. The system of claim 4, wherein the compression fabric provides a pressure of between 20 mm Hg to 30 mm Hg.

7. The system of claim 4, wherein the compression fabric provides a pressure of between 30 mm Hg to 40 mm Hg.

8. The system of claim 1, wherein the wearable garment comprises a whole-arm sleeve, an upper-arm sleeve, a lower-arm sleeve, a leg-worn sleeve, a vest, a headband, a glove, or a sock.

9. The system of claim 1, wherein the one or more sensors comprise one or more pressure sensors.

10. The system of claim 1, wherein the one or more sensors comprise one or more resistive sensors.

11. The system of claim 1, wherein the one or more sensors comprise one or more pneumatic sensors.

12. The system of claim 11, wherein at least one of the one or more pneumatic sensors comprises an inflatable bladder and a pressure transducer.

13. The system of claim 1, wherein the one or more sensors include multiple sensors arranged along a length of the wearable garment.

14. The system of claim 1, wherein at least one of the one or more processors is integrated with the wearable garment.

15. The system of claim 1, wherein at least one of the one or more processors is physically separate from the wearable garment.

16. The system of claim 1, wherein at least one of the one or more processors is configured to transmit the generated data to an application of a cloud computing environment.

17. The system of claim 1, wherein processing the generated data to determine the duration of use of the wearable garment is based comparing the generated data to one or more sensor thresholds associated with the one or more sensors.

18. The system of claim 17, wherein at least one of the one or more processors are configured to determine the sensor thresholds based on analysis of other data generated by the one or more sensors.

19. The system of claim 1, wherein at least one of the one or more processors are configured to process the generated data using machine learning (ML).

20. The system of claim 1, wherein at least one of the one or more processors are configured to process the generated data to evaluate performance of the wearable garment.

21. The system of claim 1, wherein at least one of the one or more processors are configured to process data generated by a plurality of wearable garments worn by different users.

22. A system for detecting use and performance of wearable garment, the system comprising:
one or more sensors integrated within with wearable garment; and
a controller coupled to at least one of the one or more sensors via respective ones of one or more conductive paths and configured to:
collect data generated by the one or more sensors; and
transmitting the collected data to a remote computing device,
wherein the remote computing device is configured to generate one or more metrics related to usage and performance of the garment based on the collected data and to display the metrics.

23. The system of claim 22, wherein the one or more sensors comprise one or more pressure sensors.

24. The system of claim 22, wherein the one or more sensors comprise one or more resistive sensors.

25. The system of claim 22, wherein the one or more sensors comprise one or more pneumatic sensors.

26. The system of claim 22, wherein the one or more sensors include multiple sensors disposed along a length of the wearable garment.

27. The system of claim 22, wherein the wearable garment comprises compression fabric.

28. The system of claim 22, wherein the wearable garment comprises a sleeve, vest, headband, glove, or sock.

29. The system of claim 22, comprising one or more processors configured to process data generated by a plurality of wearable garments worn by different users to evaluate collective performance of said plurality of wearable garments.

30. A system comprising:
one or more sensors integrated within a wearable garment, the sensors configured to generate data responsive to pressure exerted by the wearable garment on a user; and
one or more processors coupled to the one or more sensors and configured to process the generated data to evaluate performance of the wearable garment.
